(19) United States
(12) Patent Application Publication
(10) Pub. No.: US 2017/0364039 A1
(43) Pub. Date: Dec. 21, 2017

(54) SENSOR

(71) Applicant: TDK Corporation, Tokyo (JP)

(72) Inventors: Armin Schober, Munich (DE); Jani Kuivalainen, Munich (DE)

(21) Appl. No.: 15/544,210

(22) PCT Filed: Nov. 25, 2015
(86) PCT No.: PCT/EP2015/077635

§ 371 Date: Jul. 17, 2017

(30) Foreign Application Priority Data
Jan. 27, 2015 (DE) 102015101112.3

(51) Int. Cl. G04G 21/02; G01L 19/14; G01N 33/49; G04B 47/06

(52) U.S. Cl. CPC G04G 21/02; G01N 33/49; G01L 19/143; G04B 47/066

(57) ABSTRACT

A sensor having a chamber with a sensor element arranged therein comprises a first volume of air. The sensor has a tubular air supply to the chamber, the air supply comprising a second volume of air, and penetration of water through the air supply to the sensor element being prevented by the dimensions of the air supply that define the second volume.

### RELATED APPLICATIONS/PRIORITY CLAIMS

This application is a 371 U.S. national stage filing of (and claims the benefit and priority to under 35 U.S.C. §§119, 120, 364 and 365) to PCT/EP2015/077635, filed on Nov. 25, 2015 (and published as WO 2016/119945 on Aug. 4, 2016), that in turn claims priority under 35 USC §§119, 120, 364 and 365 to German Patent Application No. DE102015101112.3 filed Jan. 27, 2015, the entirety of all of which is incorporated herein by reference.

### FIELD OF THE DISCLOSURE

A sensor, in particular an ambient sensor, is provided.

### SUMMARY

Sensors, in particular ambient sensors, are increasingly being used in the area of portable electronic consumer goods, such as in cell phones, wristwatches and armbands. In particular when they are used in the area of sports, the sensor is expected to be waterproof.

Patent documents U.S. Pat. No. 6,754,137 B1, U.S. Pat. No. 5,500,835 A, DE 10 2006 056 128 B4 and EP 0 640 896 B1 respectively describe a wristwatch with a pressure sensor. To protect the sensor element from water getting in, the sensor element may be surrounded by a water-repellent gel and be separated from the ambient air by a membrane. Patent application DE 10 2006 040 665 Al discloses a pressure sensor for a vehicle in which water is prevented from getting into a housing of the sensor by a water-impermeable filter. Such sensors are often of a great overall size and/or are expensive to produce.

An object of the present invention is to provide an improved sensor. A sensor having a chamber with a sensor element arranged therein is provided, the chamber comprising a first volume of air. The sensor has a tubular air supply to the chamber, the air supply comprising a second volume of air. Apart from the tubular air supply, the chamber is closed off in an airtight manner. Consequently, there can only be an exchange of air between the chamber and the air supply. No air can otherwise escape however from the chamber or get into it. Penetration of water through the air supply to the sensor element is prevented by the dimensions of the air supply that define the second volume. In this case, the penetration of water is prevented at least up to an outside pressure that is less than or equal to the prescribed maximum outside pressure. Instead of the maximum outside pressure, a maximum water depth may also be prescribed.

In the case of such a sensor, penetration of water to the sensor element can consequently be prevented by the dimensions of the air supply, without additional barrier elements being required to protect against water getting in. In particular, there is no need for a barrier element that is arranged in a connecting path between the outside space and the sensor element. The connecting path leads in particular through the second volume of the air supply and the first volume of the chamber. A sensor no comprising such barrier element can be produced particularly inexpensively. Furthermore, miniaturization of the sensor is made possible.

For example, the sensor does not have a waterproof membrane that separates the sensor element from the outside space of the sensor. There is also no need for a barrier element in the form of a gel that for example surrounds the sensor element. Consequently, the ambient air can act directly on the sensor element, so that particularly accurate measurement is made possible.

It is for example a sensor for measuring properties of the outside surroundings, for example the ambient air. Such an ambient sensor is for example designed as a pressure sensor, in particular as a barometric pressure sensor, as an air humidity sensor, in particular as a sensor for measuring the relative humidity of the air, or as a gas sensor. A gas sensor is designed for measuring the concentration of a gas, for example carbon dioxide, carbon monoxide or ozone. In the case of such sensors, a direct interaction of the ambient air with the sensor element may be advantageous or even necessary for the measurement.

The sensor is preferably designed to be waterproof in the sense that water cannot penetrate to the water-sensitive components of the sensor. In this respect, the sensor may comprise along with the sensor element further water-sensitive components, in particular electrical components. The further components are for example surrounded by a waterproof housing.

In a preferred embodiment, the dimensions of the air supply have the effect not only of preventing water from penetrating to the sensor element but also of preventing water from getting into the chamber.

In particular, the dimensions of the air supply are chosen such that the air that is located in the chamber and in the air supply under atmospheric air pressure at sea level assumes a volume no smaller than the first volume when there is an increase in the outside pressure. The volume of the air under atmospheric air pressure is the sum of the first volume and the second volume. If the sensor is immersed in water, the air located within the chamber and the air supply is compressed to the extent that the pressure of the air corresponds to the outside pressure acting as a result of the water. Since the volume of the air is at least as large as the first volume within the chamber, the first volume is completely filled with air. Consequently, the water getting into the air supply cannot enter the chamber.

Suitable dimensions of the air supply can be derived from the ideal gas law. Under an atmospheric air pressure pₐₜ and a prescribed maximum outside pressure p_{max,} at which water should still not get into the chamber, the following condition is obtained for the ratio of the second volume V₂ to the first volume V₁:

V₂/V₁>=pₘₐₓ/pₐₜ−1

If 1 bar is set for the air pressure at sea level and 1 bar per 10 m of water depth is set for the increase in pressure under water, the following condition is obtained under normal conditions (pressure of 1 bar and temperature of 20° C.) for the volume ratios at a prescribed maximum water depth dₘₐₓ in meters:

V₂/V₁>=0.1 m⁻¹·dₘₐₓ

Consequently, for example, at a prescribed maximum water depth of 10 m, the second volume must be at least as large as the first volume. In one embodiment, the second volume is at least twice the first volume. In this case, penetration of water to the sensor element is prevented at least to a water depth of 20 m. Correspondingly, a working pressure range in which penetration of water to the sensor element is prevented extends for example at least over the range from 1 bar to 2 bar outside pressure, preferably at least over the range from 1 bar to 3 bar outside pressure.

In one embodiment, the chamber is of a cuboidal design. The chamber has for example an opening for the air supply. This is preferably the only opening in the chamber. Preferably, a connecting path from the sensor element to the outside space only passes through the chamber and the air supply.

The air supply preferably has a maximum inside diameter that is so small that, when immersed in water, the air enclosed cannot escape to the outside through the air supply. The maximum inside diameter of the air supply is preferably significantly less than the extent of the chamber perpendicularly to the inflow direction of the air. For example, the inside diameter is less than or equal to 1 mm. The length of the air supply is preferably significantly greater than an inside diameter of the air supply. The air supply has for example a length of at least 5 mm.

In one embodiment, the air supply is designed as a separate element. In particular, the air supply is formed separately from a housing of the chamber and/or is formed separately from an outer housing of the sensor. For example, the air supply is designed as a separate tube.

In one embodiment, the air supply is formed from a flexible material. This makes it possible to bend the air supply, for example when fitting into a housing, so that an adaptation to the geometrical dimensions of the sensor, in particular of an outer housing, is made possible. For example, the material of the air supply comprises silicone.

In one embodiment, the air supply is integrated in a housing of the sensor. In particular, the air supply may be formed as one part with a housing. For example, a housing is produced in an injection-molding process, the air supply being formed during the injection molding. In particular, the air supply may be formed during the injection molding as a passage through the housing. This makes particularly inexpensive production of the air supply possible.

In one embodiment, the air supply has at least one kink or bend. In this way, the required dimensions of the sensor can be kept small.

The sensor is for example designed as a barometric pressure sensor. The sensor element is for example designed as a piezoresistive or capacitive sensor element.

The sensor element is preferably of a miniaturized form. In particular, the sensor element has dimensions of just a few millimeters or less. This makes it possible to keep the first volume particularly small. In this case, the second volume, and in particular also the length of the air supply, can also be kept particularly small.

Consequently, the sensor as a whole can be of a miniaturized form. For example, it is suitable for use in a cell phone, a wristwatch or an armband.

The subjects described here are explained in more detail below on the basis of exemplary embodiments that are shown schematically and not to scale.

### BRIEF DESCRIPTION OF THE DRAWINGS

FIG. 1 shows a sensor according to one embodiment in a schematic sectional view.

### DETAILED DESCRIPTION OF ONE OR MORE EMBODIMENTS

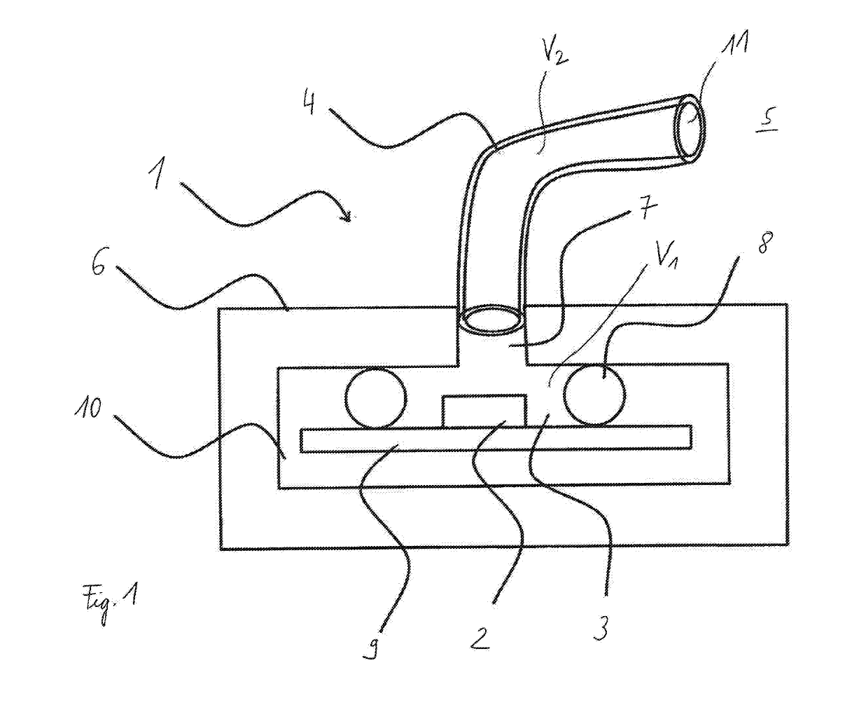

FIG. 1 shows a sensor 1, which is for example designed as a pressure sensor. The sensor 1 has a sensor element 2, which is arranged in a chamber 3. Within the chamber 3 there is a first volume V₁ of air, which partially surrounds the sensor element 2. In particular, the sensor element 2 is surrounded laterally and upwardly by the first volume V₁. The first volume V₁ is connected to an outside space 5 by a tubular air supply 4. The sensor 1 has an open structure, so that air and water can get into the air supply 4 from the outside.

The chamber 3 is partially delimited by a housing 6. The housing 6 has an opening 7, from which the air supply 4 leads away. In the embodiment shown, the air supply 4 is partially inserted in the opening 7. The air supply 4 may for example also be completely inserted in the opening 7 or outwardly adjoin the opening 7. The chamber 3 is laterally delimited by a sealing element 8, which is for example designed as a sealing ring. The sealing element 8 seals off the chamber 3 in an airtight manner. The sensor element 2 is arranged on a carrier 9, in particular a printed circuit board. The carrier 9 delimits the chamber 3 in the downward direction.

The housing 6 may enclose still further components of the sensor 1. For example, further components, in particular electronic components, are arranged in a further inside space 10, which is sealed off in an airtight manner from the chamber 3 by the sealing element 8. In particular, the air-filled volume V₁ is separated in an airtight manner from the further inside space 10. In this case, the sealing element 5 is airtight at least in the working pressure range, in particular under an outside pressure in a range between atmospheric air pressure and a prescribed maximum pressure. Consequently, an air flow from the volume V₁ is only possible into the air supply 4, but not into other regions of the sensor 1. The further inside space 10 is preferably closed off from the outside space 5 in an airtight and waterproof manner. Consequently, the air pressure in the further inside space 10 is preferably constant, for example always at normal pressure.

The first volume V₁ is connected to the outside space 5 by way of the second volume V₂ in an air-permeable manner. The air supply 4 is in this case designed such that, when the sensor 1 is immersed in water, the water can get into the air supply 4 by way of an outer opening 11. Since, apart from the outer opening 11, the air supply 4 is only connected in an air-permeable manner to the chamber 3 and, apart from the air supply 4, the chamber 3 is closed off in an airtight manner, the air enclosed in the air supply 4 and the chamber 3 cannot escape. Consequently, the air located within the air supply 4 and the chamber 3 is compressed until the pressure of the air corresponds to the outside pressure. The air supply 4 has such dimensions that, under an outside pressure that is less than or equal to a prescribed maximum pressure, the water getting in cannot enter the chamber 3.

The depicted sensor 1 is for example waterproof at least to a depth dₘₐₓ=20 m. According to the condition V₂/V₁>=0.1 m⁻¹·dₘₐₓ, the second volume is at least twice the first volume. Often, waterproofness to a depth of 50 m is desired. In this case, the second volume is at least five times the first volume.

For example, the sensor element 2 has dimensions of 2 mm×2 mm×0.8 mm (width×length×height). The chamber 3 is for example outwardly bounded by a sealing element 8 in the form of a sealing ring and has for example a lateral diameter of 2.5 mm and a height of 1 mm. The first volume V₁ within the chamber 3 is calculated from the difference between the chamber volume and the volume taken up by the sensor element as 1.7 mm³. With a prescribed maximum water depth of 20 m, a second volume V₂ of at least 3.4 mm³ is obtained. Consequently, with an inside diameter of the air supply 4 of 0.8 mm, the air supply 4 should have a length of at least 6.8 mm.

The air supply 4 has a bent shape. The air supply 4 is preferably arranged within an outer housing (not depicted) of the sensor 1. In particular, the air supply 4 does not protrude out of the outer housing. The outer housing may also be formed as one part with the housing 6. For example, the housing reaches as far as the opening of the air supply 4 to the outside space 5. The outer housing may however also be formed as a separate component from the housing 6.

In one embodiment, the air supply 4 is designed as a separate element. For example, the air supply 4 is formed by a flexible material. For example, the material comprises silicone. In an alternative embodiment, the air supply 4 is integrated in an outer housing. For example, the outer housing is injection-molded. The air supply 4 may be formed in the injection-molding process as a passage through the housing.

1. A sensor,
having a chamber with a sensor element arranged therein, the chamber comprising a first volume (V₁) of air, and having a tubular air supply to the chamber, the air supply comprising a second volume (V₂) of air, penetration of water through the air supply to the sensor element being prevented by the dimensions of the air supply that define the second volume (V₂).

2. The sensor according to claim 1, in which water is prevented from getting into the chamber by the dimensions of the air supply.

3. The sensor according to claim 1, in which the maximum inside diameter of the air supply is significantly less than the extent of the chamber (3) perpendicular to the inflow direction of the air.

4. The sensor according to claim 1, in which the second volume (V₂) is at least as large as the first volume (V₁).

5. The sensor according to claim 1, in which penetration of water to the sensor element is prevented at least to a prescribed maximum water depth dₘₐₓ in meters, the following condition applying for the ratio of the second volume (V₂) to the first volume (V₁): V₂/V₁>0.1 m⁻¹·dₘₐₓ.

6. The sensor according to claim 1, in which penetration of water to the sensor element is prevented at least to a water depth of 20 m.

7. The sensor according to claim 1, which does not have a barrier element to protect against penetration of water to the sensor element.

8. The sensor according to claim 9, which does not have a barrier element formed as a membrane.

9. The sensor according to either of claims 7, which does not have a barrier element formed as a gel.

10. The sensor according to claim 1, in which the air supply is formed from a flexible material.

11. The sensor according to claim 1, in which the air supply is integrated in a housing.

12. The sensor according to claim 11, in which the housing is injection-molded, the air supply being formed during the injection molding.

13. The sensor according to claim 1, in which the air supply has at least one kink or bend.

14. The sensor according to claim 1, in which the sensor element is surrounded laterally and upwardly by the first volume (V₁).

15. The sensor according to claim 1, which is suitable for use in a cell phone, a wristwatch or an armband.

16. The sensor according to claim 1, which is designed as a pressure sensor, as an air humidity sensor or as a gas sensor.

17. The sensor according to claim 1, wherein the length of the air supply is significantly greater than an inside diameter of the air supply.

18. The sensor according to claim 1, wherein a maximum inside diameter of the air supply is so small that, when immersed in water, enclosed air cannot escape to the outside through the air supply.
